(19) United States
(12) Patent Application Publication
(10) Pub. No.: US 2019/0298536 A1
(43) Pub. Date: Oct. 3, 2019

(54) Modular Gap Balancing Block

(71) Applicant: Howmedica Osteonics Corp., Mahwah, NJ (US)

(72) Inventors: Austin Tyler Ruiz, Jersey City, NJ (US); Matthew Demers, Ramsey, NJ (US); Shelby Carter, Austin, TX (US)

(21) Appl. No.: 16/365,895

(22) Filed: Mar. 27, 2019

Related U.S. Application Data
(60) Provisional application No. 62/648,616, filed on Mar. 27, 2018.

(51) Int. Cl. A61F 2/38; A61B 17/15; A61F 2/46; A61B 17/17

(52) U.S. Cl. CPC A61F 2/3859; A61B 17/155; A61F 2/0805; A61B 17/1764; A61B 17/157; A61F 2/4657

(57) ABSTRACT

A method of preparing a femur for receipt of a prosthesis, includes selecting an assessment device from a plurality of devices, each of the devices include first and second contact surfaces positioned at different elevations from a bottom surface of the device; inserting the device into a knee joint such that the bottom surface of the device contacts a resected surface of a tibia, the first contact surface contacts a first condylar surface, and the second contact surface contacts a second condylar surface, the first and second contact surfaces have a fixed relationship relative to the bottom surface while disposed within the joint; determining a condylar angle of a condylar axis defined by the first and second condylar surfaces based on a known offset distance between the first and second contact surfaces; and resecting the first and second condylar surfaces along a plane based on the condylar angle.

### CROSS-REFERENCE TO RELATED APPLICATIONS

This application claims the benefit of the filing date of U.S. Provisional Application No. 62/648,616, filed Mar. 27, 2018, the disclosure of which is hereby incorporated herein by reference.

### BACKGROUND OF THE INVENTION

Total knee arthroplasty (“TKA”) or total knee replacement is a common orthopedic procedure in which damaged or diseased cartilage and/or bone of the knee is replaced with prosthetic components. Prior to implanting such prosthetic components, a surgeon resects a portion of the patient's native bone in order to shape the bone to receive the prosthetic components. For example, a surgeon might make one or more planar cuts at a distal end of a femur and proximal end of a tibia so that corresponding surfaces of femoral and tibial prosthetic components can be respectively attached thereto.

Each individual cut of bone is carefully made. Once native bone is resected from a joint, it is gone forever. In addition, the amount of bone resected and the final geometries of the resected bone significantly influence the fit and alignment of the prosthetic components. Improper fit and/or alignment can result in instability of the joint, increased risk of bone fracture and/or component failure, pain, and reduced range of motion.

Multiple resection philosophies/techniques have emerged over the years to help ensure proper fit and alignment of the prosthetic components comprising the artificial joint. One such technique is a gap balancing technique in which flexion and extension gaps between the femur and tibia are balanced so that tension in the collateral ligaments is consistent during rotation of the joint through flexion and extension. This is important as imbalanced gaps can result in instability of the knee, reduced range of motion, and/or pain.

Another consideration for proper fit and alignment is achieving an appropriate prosthesis joint-line between tibial and femoral prosthetic components when finally implanted. An improper or malaligned prosthesis joint-line can result in an awkward gait and increased wear of the TKA prosthesis. Whether the desired prosthesis joint-line be varus, valgus, or neutral, it is common to assess the native articular surfaces relative to a reference, such as a long axis of the femur and/or tibia, in order to determine the appropriate angle of resection to achieve the desired joint-line and ensure proper communication between the prosthetic components.

Such assessment can be performed through pre-operative planning and through referencing instruments intraoperatively. Current referencing instruments that are used to assess flexion and extension gaps and native articular surfaces are typically dynamic systems which generally include linearly moving and rotating plates that contact both the femur and tibia. However, the moveable components of such dynamic systems are prone to failure and inaccuracies due to movement of the patient or instability of the patient's joint. Additionally, such dynamic systems typically require retractors and other forms of exposure that can stress soft tissue, such as a patellar tendon. Thus, further improvements are desirable.

### BRIEF SUMMARY OF THE INVENTION

Described herein are devices, systems, and methods for performing a TKA. In particular, a modular assessment assembly is disclosed that includes a rotation block and a spacer shim. The rotation block and spacer shim are connectable such that the spacer shim increases the overall thickness of the rotation block by a predetermined increment, which is associated with an incremental size of a TKA prosthetic component. In this regard, the rotation block, either alone or in combination with the spacer shim, can be used to assess the flexion and extension gaps of a patient's knee joint including when such knee joint includes unresected articular surfaces.

In addition, the rotation block has contact pads that include bone contact surfaces which may be offset relative to each other in a direction coincident with the rotation block's thickness. The offset distance between the contact surfaces of the contact pads is associated with a rotational offset of a native condylar axis relative to a reference axis. In this regard, the rotation block, either alone or in combination with the spacer shim, can be used to assess a varus/valgus angle of a patient's distal condylar axis and/or internal/external rotational angle of a patient's posterior condylar axis relative to the reference axis. Such reference axis may assume a neutral orientation perpendicular to a tibial shaft axis or some other predetermined orientation relative to the shaft axis. Thus, the system provides a static, modular platform through which a native, unresected femur can be assessed prior to component sizing.

Also disclosed are kits that include a plurality of rotation blocks and a plurality of spacer shims Each of the rotation blocks of the kit may be associated with a different angular orientation of a condylar axis. In this regard, each rotation block may include contact surface offsets associated with respective femoral rotational angles of 0, 1.5, 3, 4.5, and 6 degrees, for example. In addition, the spacer shims of the kit may have thicknesses that increase in 1 mm increments from a spacer shim that is 1 mm to a spacer shim that is 9 mm, and each rotation block of the kit may have a nominal thickness of 7 mm Thus, the rotation block and spacer shim combination can assess extension/flexion gaps from 7 to 16 mm.

In a first aspect of the present disclosure, a method of evaluating a knee joint for resecting the same to receive a prosthetic component includes selecting a first rotation block from a plurality of rotation blocks. The plurality of rotation blocks each have first and second upper surfaces and a lower surface disposed opposite the first and second upper surfaces. The first and second upper surfaces are offset relative to each other a distance extending in a direction transverse to the lower surface. The distance differs between each of the plurality of rotation blocks. The first and second upper surfaces define an offset axis having a fixed angular orientation relative to an axis defined by the lower surface. The fixed angular orientation differs between each of the plurality of rotation blocks. The method also includes inserting the first rotation block into a gap between a resected proximal surface of a tibia and unresected condylar surfaces of a femur such that the lower surface contacts the resected proximal surface of the tibia, the first upper surface contacts a first unresected condylar surface of the femur, and the second upper surface contacts a second unresected condylar surface of the femur. The method further includes evaluating correspondence between the offset axis of the first rotation block with a condylar axis defined by the first and second condylar surfaces.

In addition, the method may further include evaluating collateral ligament tension while the first rotation block is disposed within the gap. The method may also include selecting a first spacer from a plurality of spacers if it is determined that the collateral ligament tension is too loose. The plurality of spacers may each have an upper surface and a lower surface and a thickness defined therebetween. The thickness differs between each of the plurality of spacers by predetermined increments. Also, the method may include: connecting the first spacer to the first rotation block such that the upper surface of the first spacer is positioned adjacent the lower surface of the first rotation block and so that the lower surface of the first spacer assumes the lower surface of the first rotation block for contact with the resected proximal surface of the tibia, inserting the first rotation block and spacer into the gap, and reevaluating the collateral ligament tension while the first rotation block and spacer are disposed within the gap. The connecting step may include one of inserting pegs of the first spacer into corresponding openings in the rotation block and sliding a dovetail of the first spacer into a correspondingly shaped groove extending into the lower surface of the first rotation block. Even further, the method may include disconnecting a second spacer of the plurality of spacers from the rotation block prior to connecting the first spacer to first the rotation block.

Continuing with this aspect, the method may include selecting a second rotation block from the plurality of rotation blocks and repeating the inserting and evaluating steps when it is determined that there is no correspondence between the offset axis of the first rotation block and the condylar axis. The method may also include calibrating a bone preparation instrument based on the fixed angular orientation when it is determined that there is correspondence between the offset axis and condylar axis. Even further, the method may include connecting a handle to the first rotation block prior to inserting the first rotation block into the gap, and disconnecting the handle from the first rotation block once the first rotation block is disposed in the gap. The condylar axis may be a posterior condylar axis of unresected posterior condyles. Alternatively, the condylar axis may be a distal condylar axis of unresected distal condyles.

Furthermore, the lower surface and the first and second upper surfaces of each of the plurality of rotation blocks may be planar. The first and second upper surfaces of each of the plurality of rotation blocks may have a surface area defined by a standard deviation of a dataset comprised of distances between tangent points of lateral and medial condyles of a population of femurs. The standard deviation may be a second standard deviation.

In another aspect of the present disclosure, a method of preparing a femur for receipt of a prosthesis includes selecting a first rotation block from a plurality of rotation blocks. The plurality of rotation blocks may each have first and second upper surfaces and a lower surface disposed opposite the first and second upper surfaces. The first and second upper surfaces are offset relative to each other a distance extending in a direction transverse to the lower surface. The distance differs between each of the plurality of rotation blocks. The first and second upper surfaces define an offset axis that has a fixed angular orientation relative to an axis defined by the lower surface. The fixed angular orientation differs between each of the plurality of rotation blocks. The method also include selecting a first spacer from a plurality of spacers. The plurality of spacers each have an upper surface and a lower surface and a thickness defined therebetween. The thickness differs between each of the plurality of spacers by predetermined increments. The method also includes connecting the first spacer to the first rotation block such that the upper surface of the first spacer is positioned adjacent the lower surface of the first rotation block. Even further, the method includes inserting the first rotation block into a gap between a resected proximal surface of a tibia and unresected condylar surfaces of a femur such that the lower surface of the first spacer contacts the resected proximal surface of the tibia, the first upper surface of the first rotation block contacts a first unresected condylar surface of the femur, and the second upper surface of the first rotation block contacts a second unresected condylar surface of the femur. Also included in the method is evaluating gap tension and correspondence between the offset axis of the first rotation block with a condylar axis defined by the first and second condylar surfaces. If the gap tension was determined to be too loose or too tight, selecting a second spacer from the plurality of spacers and repeating the connecting, inserting, and evaluating steps. If the condylar axis was determined to not correspond with the offset axis of the first rotation block, selecting a second rotation block and repeating the connecting, inserting, and evaluating steps. The method also includes resecting the first and second condylar surfaces along a resection plane at an angle from the condylar axis substantially equal to the fixed angular orientation of one of the rotation blocks determined to have a corresponding offset axis with the condylar axis.

In addition, the resecting step may include resecting the first and second condylar surfaces at a depth corresponding to a combined thickness of a finally selected spacer and rotation block.

In a further aspect of the present disclosure, a method of preparing a femur for receipt of a prosthesis includes selecting a static assessment device from a plurality of static assessment devices. Each of the static assessment devices have first and second femoral contact surfaces positioned at different elevations from a bottom surface of the static assessment device. The method also includes inserting the static assessment device into a gap between a femur and tibia such that the bottom surface of the static assessment device contacts a resected surface of a proximal tibia, the first femoral contact surface contacts a first condylar surface, and the second femoral contact surface contacts a second condylar surface. The first and second femoral contact surfaces have a fixed relationship relative to the bottom surface while disposed within the gap. Even further, the method includes determining a condylar angle of a condylar axis defined by the first and second condylar surfaces based on a known offset distance between the first and second femoral contact surfaces, and resecting the first and second condylar surfaces along a resection plane based on the determined condylar angle.

In addition, the static assessment device may include a rotation block and a spacer removeably connected to the rotation block. The rotation block may include the first and second femoral contact surfaces, and the spacer may comprise the bottom surface. The method may also include connecting a handle to the static assessment device prior to inserting the first rotation block into the gap, and disconnecting the handle from the static assessment device once the first rotation block is disposed in the gap.

### BRIEF DESCRIPTION OF THE DRAWINGS

The features, aspects, and advantages of the present invention will become better understood with regard to the following description, appended claims, and accompanying drawings in which:

FIG. 1 is a front exploded view of a static gap assessment system according to an embodiment of the present disclosure.

FIG. 2A is a rear perspective view of a rotation block of the static gap assessment system of FIG. 1.

FIG. 2B is a front elevational view of the rotation block of FIG. 2A.

FIG. 2C is a top view of the rotation block of FIG. 2A

FIG. 3A is a front perspective view of a spacer shim of the static gap assessment system of FIG. 1.

FIG. 3B is a front view of the spacer shim of FIG. 3A.

FIGS. 5-8 illustrates a method of using the static gap assessment system according to an embodiment of the present disclosure.

FIG. 9A is a front perspective view of a static gap assessment system according to another embodiment of the present disclosure and as partially assembled.

FIG. 9B is a front perspective view of the static gap assessment system of FIG. 9A as fully assembled.

FIG. 10 is a front perspective view of a rotation block of a static gap assessment system according to a further embodiment of the present disclosure.

### DETAILED DESCRIPTION

As used herein, unless stated otherwise, the term “proximal” means closer to the heart, and the term “distal” means further from the heart. The term “anterior” means toward the front part of the body or the face, the term “posterior” means toward the back of the body. The term “medial” means closer to or toward the midline of the body, and the term “lateral” means further from or away from the midline of the body. The term “inferior” means closer to or toward the feet, and the term “superior” means closer to or toward the crown of the head. The term “flexion/extension (“F/E”) gap” refers to the gap formed between a femoral condyle and a surface of a proximal tibia when the knee joint is in flexion (about 90 degrees) and full extension. As used herein, the terms “about,” “generally,” and “substantially” are intended to mean that slight deviations from absolute are included within the scope of the term so modified.

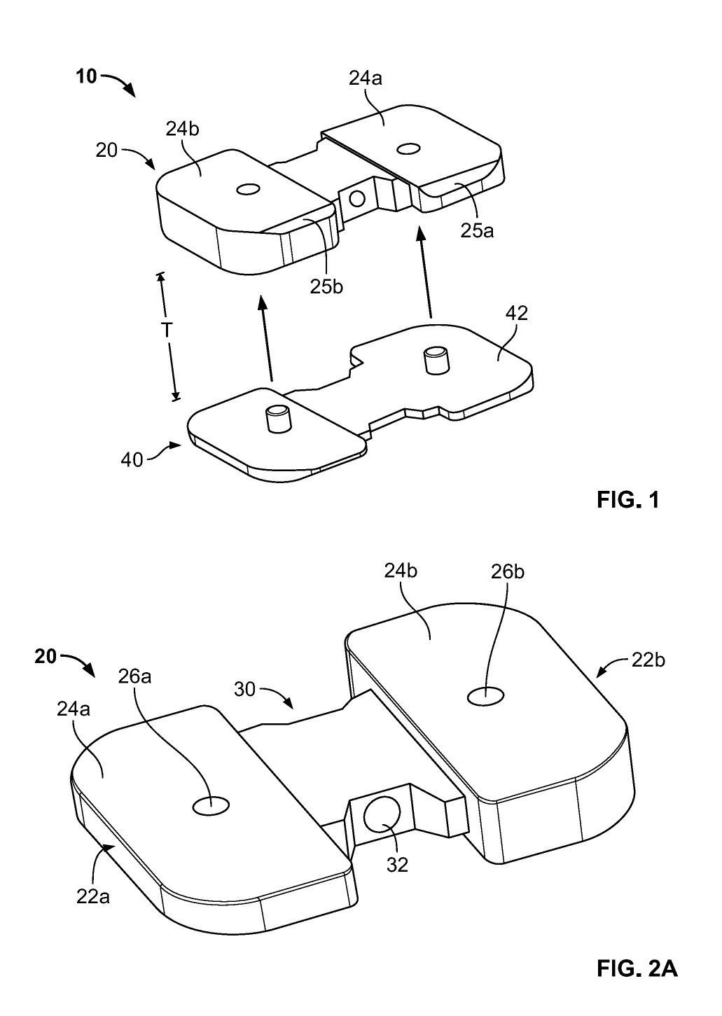

FIG. 1 depicts a modular static gap assessment assembly 10 that includes a rotation block 20 and a spacer shim 40. As described in more detail below, rotation block 20 and spacer shim 40 are connectable to each other and are configured to be inserted into a flexion and/or extension gap between a femur and a tibia for assessing the F/E gaps and orientation of the femur's condylar axes.

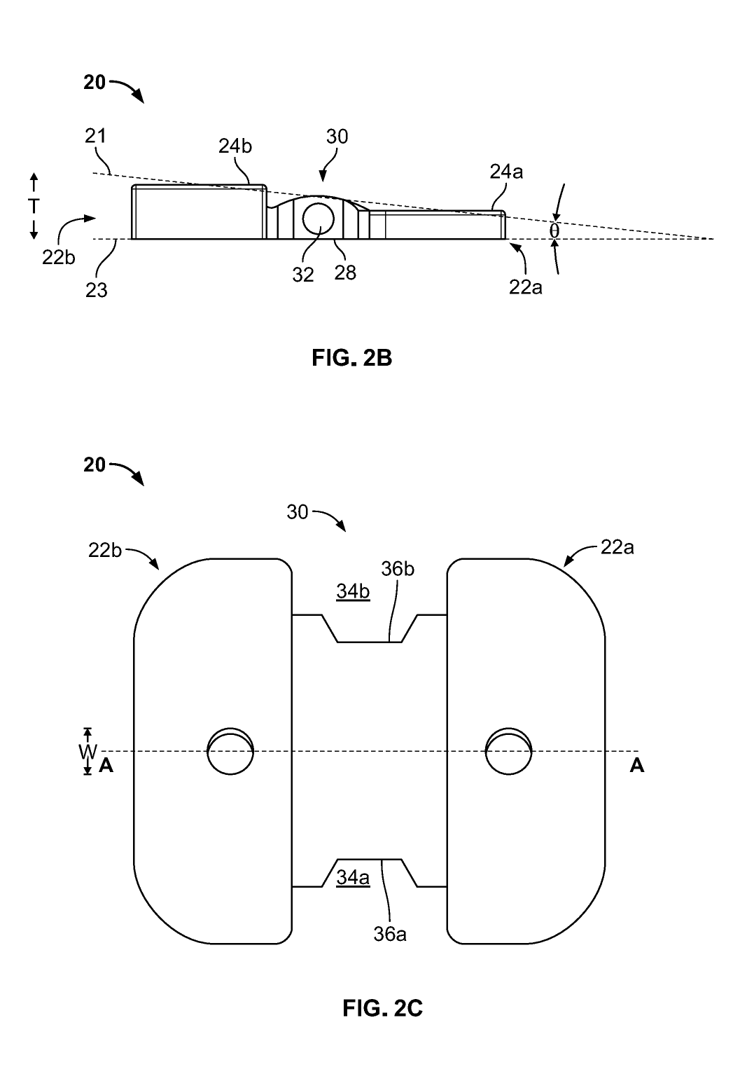

FIGS. 2A-2C depict rotation block 20 which includes a first contact pad 22a, a second contact pad 22b, and an intermediate section 30 disposed between first and second contact pads 22a-b. First contact pad 22a includes a first femoral contact surface or first upper surface 24a, while second contact pad 22b includes a second femoral contact surface or second upper surface 24b. Rotation block 20 includes a bottom surface 28 disposed opposite first and second contact surfaces 24a-b that spans first and second contact pads 22a-b and intermediate section 30.

As best shown in FIG. 2B, first contact surface 24a is elevated relative to bottom surface 28 lower than that of second contact surface 24b. In this regard, the thickness of rotation block 20 at first contact pad 22a is smaller than that at second contact pad 22b where the thickness is in a direction identified as “T” in FIG. 2B. First and second contact surfaces 24a-b and bottom surface 28 are each planar surfaces, but may have chamfered edges 25a-b as shown in FIG. 1 to facilitate insertion into a knee joint gap. Also, bottom surface 28 defines a reference axis 23, and first and second contact surfaces 24a-b define an offset axis 21 that intersects corresponding points on first and second offset surfaces 24a-b and is oriented relative to reference axis 23 by an offset angle θ. Offset angle θ is a function of an offset distance in the direction T between first and second contact surfaces 24a-b. The greater the offset distance, the larger the offset angle θ. Thus, the offset angle θ may be predetermined and can be used to determine a condylar angle of condylar surfaces which contact first and second contact surfaces 24a-b. In other words, when prominences of a medial and lateral condyle uniformly contact first and contact surfaces 24a-b, respectively, such that surfaces 24a-b are tangent to the prominences, it can be determined that a condylar axis defined by such prominences is coincident with offset axis 21 and, therefore, rotated angle θ relative to reference axis 23. Rotation block 20 is a static, monolithic structure and can be provided with any number of different offset angles θ such as any angle between 0 and 9 degrees, for example, but preferably in 1.5 degree increments such as 0, 1.5, 3, 4.5, and 6 degrees.

As mentioned above, rotation block 20, as shown, is thicker at second contact pad 24b than at first contact pad 24a. In the embodiment mentioned above where the offset angle θ is zero degrees, first and second contact pads 22a-b will have the same thicknesses. However, regardless of the differences in thicknesses between first and second contact pads 22a-b, rotation block 20 is designated as having a nominal thickness. Such nominal thickness is associated with a thickness of a flexion or extension gap. Moreover, such nominal thickness corresponds to a particularly sized prosthetic component, such as a tibial insert (not shown). Such tibial inserts are known in the art and are typically polymeric components that are provided in multiple sizes and are sandwiched between a femoral prosthetic component and tibial baseplate prosthesis. By way of example, rotation block 20 may have a nominal thickness of 6 to 10 mm Thus, a rotation block 20 having a 7 mm nominal thickness may correspond to a 7 mm tibial insert.

Intermediate section 30 is disposed between contact pads 22a-b and generally provides a connection location for an inserter instrument and also provides clearance for femoral condyles to contact surfaces 24a-b. However, it is contemplated that rotation block 20 may not include an intermediate section 30 and that in such embodiment first and second contact pads 22a-b may abut each other. As best shown in FIG. 2C, intermediate section 30 has a width extending in the direction W which spans from a front end to a back end of rotation block 20 and is smaller than the widths of contact pads 22a-b. In this regard, contact pads 22a-b and intermediate section 30 define cutouts 34a-b at the front and back ends of rotation block 20. In addition, intermediate section 30 includes sidewalls each with a tapered notch 36a-b tapering inwardly toward axis A-A at both the front and back ends of intermediate section 30. Such tapered notches 36a-b are trapezoidal in shape and are configured to mate with a correspondingly shaped end of an inserter instrument, as described below. However, other shapes are contemplated, such as semicircular, triangular, rectangular, and the like, for example. Moreover, to help facilitate connection with an inserter instrument, an opening 32 extends through the sidewalls of the intermediate section 30 at notches 36a-b. Such opening 32 is preferably threaded at each end of rotation block 20 so as to threadedly receive a threaded projection of an inserter instruments, as described in more detail below.

Rotation block 20 is universal such that it is configured to be applicable to at least 95% of the patient population. This is achieved by configuring the surface areas of the respective contact surfaces 24a-b and their relative distance across the intermediate section 30 via a statistical analysis of a database that includes bone data for a diverse population of human adult femurs. In an example, an analysis of 937 adult femurs, both male and female ranging from 18 to 109 years old, may be performed in order to determine the 50^{th} percentile value for a distance between tangent points of medial and lateral posterior condyles of a femur. This calculation may be completed to two standard deviations in order to encompass 95% of the patient population. First and second contact surfaces 24a-b are then configured so as to encompass the plotted values up to the second standard deviation. Such calculation can also be performed on tangent points of medial and lateral distal condyles of the femurs of a diverse population to ensure first and second contact surfaces 24a-b would also encompass at least 95% of the patient population.

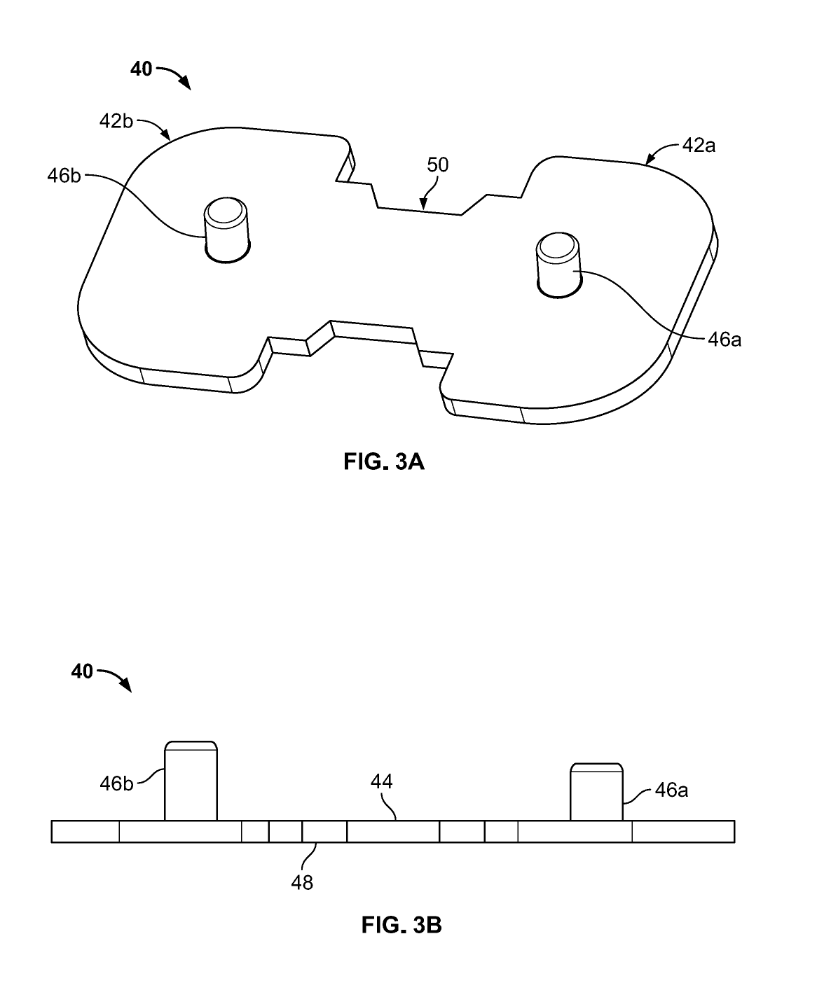

In situations where the nominal thickness of rotation block 20 is not sufficient to approximate an F/E gap, a spacer shim 40 may be connected thereto to increase its thickness. FIGS. 3A-3B depict spacer shim 40 which includes first and second wings 42a-b and an intermediate section 50 disposed therebetween. As best shown in FIG. 3A, first and second wings 42a-b and intermediate section 50 have the same profile as rotation block 20. This allows an inserter instrument to connect to the entire assembly 10 even when spacer shim 40 is connected to rotation block 20 and to assume the same footprint within the flexion or extension gap.

Spacer shim 40 also includes an upper surface 44 and a lower surface 48. Upper and lower surfaces 44, 48 are planar and extend across the entirety of spacer shim 40. However, lower surface 48 may include chamfers at the front and back end of wings 42a-b to facilitate insertion into a gap between a tibia and femur. A thickness of spacer shim 40 spans between upper and lower surfaces 44, 48. Spacer shim 40 is configured to increase the overall thickness of rotation block 40 by a predetermined amount in order to facilitate assessment of a flexion and/or extension gap. In this regard, the thickness of spacer shim 40 may be provided in 1 to 2 mm increments so that a plurality of spacers 40 can include spacers ranging from 1 or 2 mm to 10 mm in total thickness.

Posts or pegs 46a-b extend from upper surface 44 at first and second wings 42a-b. Such posts 46a-b are configured to be received within openings 26a-b of rotation block 20 so as to releasably connect spacer shim 40 to rotation block 20. In this regard, posts 46a-b may be tapered to facilitate a taper lock connection or may be configured for a snap-fit connection, for example.

As depicted in FIG. 1, rotation block 20 can be connected to spacer shim 40 such that pegs 46a-b are received within respective openings 26a-b and upper surface 42 of spacer shim 40 contacts and co-aligns with bottom surface 28 of rotation block 20. As mentioned above, rotation block 20 has a nominal thickness, which may be 5-8 mm Thus, connecting spacer shim 40 to rotation block 20 increases the nominal thickness of rotation block 20 by an incremental amount equal to the thickness of spacer shim 40. Thus, when a spacer shim of 1 to 10 mm thick is connected to a rotation block 20 having a nominal thickness of 7 mm, the overall assembly 10 assumes a nominal thickness of 8 to 17 mm.

In keeping with the universal character of assembly 10, assembly 10 is symmetric about axis A-A shown in FIG. 2C. Indeed, due to this symmetry, assembly 10 can be used in both right and left knees of a patient by turning assembly 10 around so that, for example, its leading end when inserted into a right knee becomes its trailing end when inserted into a left knee.

When entering into a TKA procedure, a surgeon typically does not know with certitude the orientations of the patient's condylar axes or the thickness of their flexion and extension gaps. Such determinations are generally made during the surgical procedure. In this regard, a kit containing a plurality of rotation blocks 20 and a plurality of spacer shims 40 may be provided to account for any one of multiple scenarios that may be presented. Such kit may be provided with a plurality of rotation blocks 20 that each have the same nominal thickness but each have a different offset axis 21 such that any one of the rotation blocks 20 may match a condylar axis orientation of the patient. In addition, such kit may also be provided with a plurality of spacer shims 40 that each have a different thickness so that a selected rotation block 20 either alone or in combination with one of the spacer shims 40 can match a thickness of a flexion or extension gap of the patient. This allows for a significant number of combinations to account for a multitude of different scenarios while keeping the number of instruments to a minimum.

More specifically, a first exemplary kit embodiment may include a plurality of rotation blocks 20 having offset angles differing by 1.5 degrees and each having a nominal thickness of 7 mm. For example, the kit may include rotational blocks 20 having offset angles of 0, 1.5, 3, 4.5, and 6 degrees, respectively. The kit may also include spacer shims 40 having differing thicknesses by increments of 1 mm. For example, the kit may include spacer shims 40 having thicknesses of 1, 2, 3, 4, 5, 6, 7, 8, and 9 mm, respectively. Thus, such kit embodiment can be used to assess gaps that are 7 to 16 mm and condylar axes that are rotated 0 to 6 degrees.

Other kit embodiments can forgo spacer shims 40 and instead include a plurality of rotation blocks 20 that have differing nominal thicknesses and differing offset angles θ. For example, in a second exemplary kit embodiment, a plurality of rotation blocks 20 include a first set of rotation blocks 20 having an offset angle of 1.5 degrees and having differing nominal thicknesses ranging from 7 to 16 mm, a second set of rotation blocks 20 having an offset angle of 3 degrees and having differing nominal thicknesses ranging from 7 to 16 mm, and so forth. However, such second kit that is assembled to assess the same range of scenarios as the first kit embodiment above would include more total components in the kit.

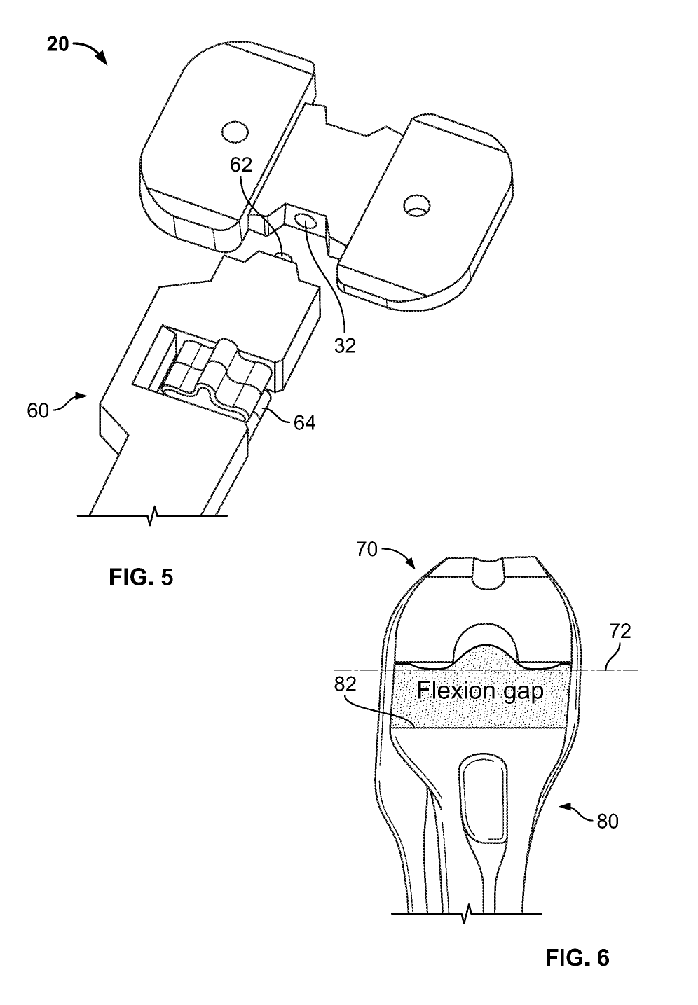

A universal inserter instrument 60 may also be included in the kit described above. FIG. 5 best depicts inserter instrument 60 configured to mate with each rotation block 20 within the kit. Inserter instrument 60 includes an insertion end that is correspondingly shaped to be received in the tapered notches 36a-b of rotation block 20. The insertion end of instrument 60 itself has a thickness which is preferably smaller than at least one of contact pads 22a-b. Moreover, it is preferable that, when inserter 60 is engaged to block 20, no part of the insertion end projects lower than bottom surface 28 so that inserter 60 does not impinge on a tibia during insertion.

In addition, universal inserter instrument 60 includes a threaded projection 62 connected to a thumbwheel 64 is configured to be received in threaded opening 32 and extends from the insertion end. Threaded projection 62 includes threads that engage with threads of opening 32. Such threads may have a pitch configured to limit the number of turns of thumbwheel 64 to securely engage block 20. For example, threaded projection 62 may have a thread pitch configures such that it would take no more than four complete turns of thumbwheel 64 to securely engage projection 62 with opening 32. This helps minimize the time needed for instrument assembly and also helps the operator know when there is secure engagement so as to not over or under tighten.

A method of preparing a right leg femur 70 using assembly 10 to assess a flexion gap and posterior condylar axis is illustrated in FIGS. 5-8. In the method, a tibia 80 is resected so as to form a proximal resected surface 82. Such resection may be made so that the proximal resected surface 82 is perpendicular to a tibial shaft axis, for example. A first rotation block 20 is then selected from a kit containing a plurality of rotation blocks 20, such as the first kit embodiment described above. Such first rotation block 20 may be selected as a best initial approximation of a posterior condylar axis 72. The first rotation block 20 is then connected to inserter instrument 60 such that the insertion end of inserter 60 is positioned within the appropriate notch 36a-b corresponding to the particular leg (right or left) the procedure is being performed upon. Thumbwheel 64 is rotated so that the threaded projection 62 threadedly engages opening 32 to secure inserter 60 to first rotation block 20.

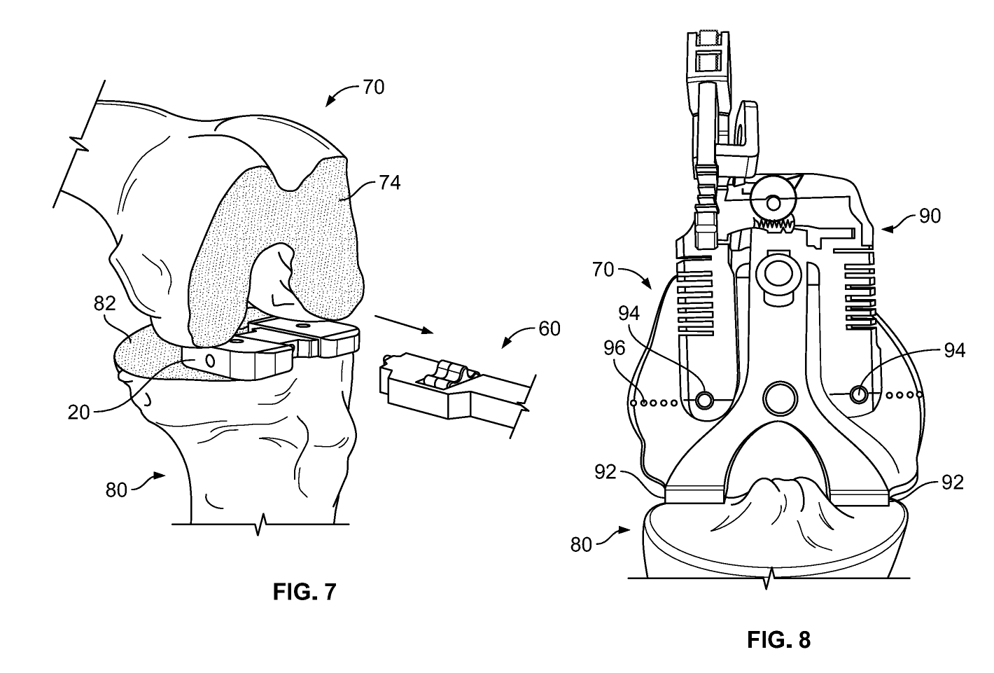

Thereafter, tibia 80 is rotated so that the knee joint is in flexion and the unresected posterior condyles face proximal resected surface 82 of tibia 80, as shown in FIG. 6. First rotation block 20 is then inserted into the flexion gap so that the bottom surface 28 thereof rests on proximal resected surface 82 of tibia 80 and so that lateral posterior condyle rests on first contact surface 24a and medial posterior condyle rests on second contact surface 24b, as best shown in FIG. 7. It should be understood that it is not required that first rotation block 20 be inserted into the flexion gap alone before being connected to a spacer shim 40. Indeed, the method may begin by connecting first rotation block 20 with a spacer shim 40 and inserting the assembly 10 into the flexion gap so that bottom surface 48 of shim 40 contacts tibia 80.

Inserter 60 is then removed by unthreading threaded projection 62 from opening 32. This facilitates an unobstructed view of rotation block 20 within the flexion gap. At this point, rotation block 20 provides a static platform that allows for unimpeded assessment of the flexion gap and posterior condylar axis 72. In addition, it allows the tissues surrounding the knee joint to be relaxed, rather than tensioned by retractors and the like, to obtain an accurate assessment. Once instrument 60 is removed, operator assesses the flexion gap tension. In addition, operator assesses the posterior condyles and posterior condylar axis 72 for uniform conformity with first and second contact surfaces 24a-b of rotation block 20 and its offset axis 21.

If it is determined that the tension in the collateral ligaments is too tight, the surgeon may release one of the collateral ligaments according to known methods. The operator may instead remove a spacer shim 40 from first rotation block 20 in the event a spacer shim 40 was connected thereto prior to insertion. If it is determined that the tension is too loose, then the surgeon may select a spacer shim 40 for connection to rotation block 20 in order to increase its thickness. In addition, if it was determined that the posterior condylar axis 72 did not uniformly conform to rotation block 20, then a second rotation block 20 having a different offset angle 21 may also be selected. If a spacer 40 or different spacer 40 is needed and/or a different rotation block 20 is needed to continue the assessment, operator may then connect inserter instrument 60 to first rotation block 20 and remove it from the flexion gap. A newly selected second rotation block 20 and spacer 40 are then connected together and to inserter 60. Insertion and assessment is then repeated until it is determined that a final selected assembly 10 best conforms to the flexion gap and posterior condyles. Thus, assessment is performed iteratively until the operator is satisfied with the conformity.

Once there is conformity, the known nominal thickness of the finally selected assembly 10 and offset angle θ of the finally selected rotation block 20 is used in subsequent steps of the procedure. For instance, the known thickness of assembly 10 may be used to select correspondingly sized prosthetic components, such as a correspondingly sized tibial insert, for filling the flexion gap after the posterior condyles are resected. Moreover, the nominal thickness of the finally selected assembly 10 may dictate the depth of resection of the posterior condyles.

In addition, as shown in FIG. 8, a separate femoral preparation instrument 90 may be calibrated based on the known offset angle θ of the finally selected rotation block 20. More specifically, an anterior-posterior sizer (“A/P sizer”) 90 may be adjusted so that drill holes 94 thereof are angled relative to condylar contact legs 92 based on the known offset angle θ. For example, if the finally selected rotation block 20 has an offset angle of 3 degrees, an axis 96 intersecting drill holes 94 is adjusted so as to be angled 3 degrees relative to a contact axis defined by posterior condylar contact surfaces of the legs 92. Pins may then be drilled into the bone through drill holes 94. Examples of other A/P sizer instruments that may be used in association with assessment assembly are disclosed in U.S. Publication No. 2017/0100132 (“the '132 Publication”), the disclosure of which is incorporated by reference herein in its entirety.

Drill holes 94 may then be used by other instruments, such as a 4-in-1 cutting guide and the like to guide a saw blade to cut the posterior condyles for the femoral component prosthesis. Exemplary instrumentation for performing a posterior condylar resection based on the above mentioned drill holes can also be found in the aforementioned and incorporated '132 Publication. Thus, the assessment performed by assembly 10 sets the stage for resecting the posterior condyles so as to achieve the desired alignment of the posterior resection relative to the proximal tibial resection 82.

While the above method describes using system 10 to assess a flexion gap and an angular orientation of a posterior condylar axis 72 of native posterior condyles relative to a resected proximal tibial plateau 82, it should be understood that system 10 can also asses an extension gap and distal condylar axis of unresected distal condyles. In this regard rotation block 20 may be used to identify a varus/valgus angle of distal condyles of a femur and may also be used alone or in conjunction with spacer shim 40 to assess the extension gap. Such a method of assessing the flexion gap and distal condylar axis is performed substantially the same as previously described with the difference being that assembly 10 is inserted into the extension gap rather than the flexion gap.

Assessing both the flexion and extension gaps using system 10 allows an operator to balance the flexion and extension gaps. For example, where the thicknesses of the flexion gap and extension gap differ as determined by the nominal thickness of rotation block 20 including any spacer shims 40, the operator may elect to resect more bone from the distal femur by an amount corresponding to this difference so that the gaps will be substantially equal once the prosthesis is implanted. For example, where the flexion gap is 1 mm thicker than the extension gap, the operator may resect the distal femur one additional millimeter from a minimum starting resection depth.

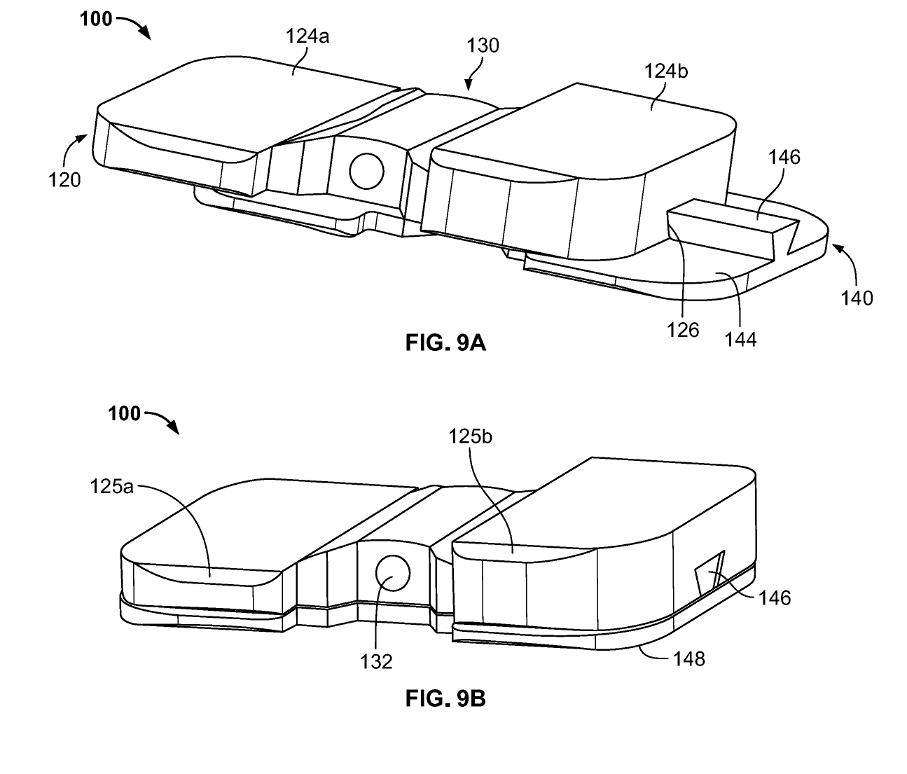

FIGS. 9A-9B depict another embodiment modular gap assessment system 100. For ease of review, like elements are accorded like reference numerals to that of system 10, but within the 100-series of numbers. For instance, system 100 includes a rotation block 120 and a spacer shim 140. Rotation block 120 includes contact surfaces 124a-b and intermediate section 130, and spacer shim 140 includes an upper surface 144 and lower surface 148 defining a thickness therebetween. However, system 100 differs from system 10 in the connection mechanism between rotation block 120 and spacer shim 140. In this regard, the connection mechanism of system 100 includes a dovetail 146 and groove 126 connection in which spacer shim 140 includes the male dovetail 146 and the rotation block 120 includes the correspondingly shaped female groove 126. While it is contemplated that spacer shim 140 includes groove 126 and rotation block 120 includes dovetail 146, the configuration as shown is preferable as it allows rotation block 120 to be used alone in a knee joint and to contact a planar resected surface of a proximal tibia. This configuration also allows spacer shim 140 to have a smaller minimum thickness as groove 126 for dovetail 146 would require spacer shim 140 to have sufficient thickness to allow for such a groove 126.

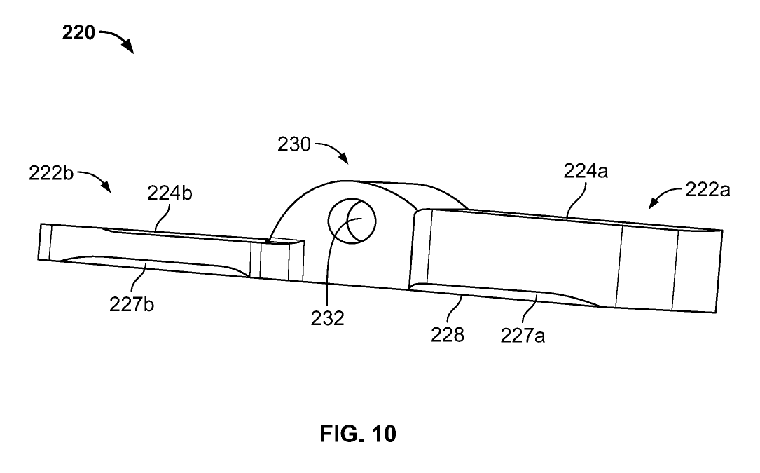

FIG. 10 depicts a rotation block 220 according to a further embodiment of the present disclosure. Rotation block 220 is similar to blocks 20 and 120 in that it can be configured to be connected to either shim 40 or 140 to increase its thickness. In addition, rotation block 220 includes contact pads 222a and 222b with respective first and second contact surfaces 224a-b and an intermediate section 230 positioned between contact pads 222a-b. However, block 220 differs in that the intermediate section 230 has a greater thickness than that of either contact pad 222a or 222b, whereas intermediate sections 30 and 130 have smaller thicknesses than at least one their respective pads 22a-b, 222a-b. In this regard, intermediate section 230 has a maximum height relative to bottom surface 228 greater than that of either contact surface 224a or 224b. This helps the operator identify the centerline of rotation block 220 when placed into a knee joint so as to ensure proper bone contact with surfaces 224a-b. Moreover, as shown, intermediate section 230 is cylindrically curved. This helps intermediate section 230 fit within an intercondylar notch of a femur, such as the intercondylar notch 74 shown in FIG. 6. The height of intermediate section 230 further helps prevent rotation block 220 from moving medially-laterally within the knee joint as such intermediate section 230, due to its proud height, abuts either a lateral or medial condyle of a femur preventing it from moving during use. In addition, rotation block 220 includes chamfers 227a-b at the bottom surface of the leading ends of both contact pads 222a and 222b. This helps with insertion of rotation block 220 into knee joint where a spacer shim is not used.

In addition, while it is described above that upper first and second contact surfaces 24a-b, 124a-b and lower surfaces 28, 128, 48, 148 are planar, it is contemplated that one or more of such surfaces may be curved. For example, lower surfaces 28, 128, 48, 148 which contact the tibial resected surface may be curved, such as convexly or concavely curved, where such resected surface is curved. However, it is typical to resect a proximal tibia along a plane. Thus, it should be understood that the lower bone contact surfaces of the devices described herein can be configured in any number of ways provided they conform to the proximal tibia. With regard to the upper contact surfaces, 24a-b and 124a-b, it is also preferred that such surfaces be planar so as to accommodate the vast majority of patient population and also to help ensure tangential contact with femoral condyles. However, other configurations are contemplated for tangential contact. For example, upper contact surfaces may be cylindrically convex and extend laterally-medially.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method of evaluating a knee joint for resecting the same to receive a prosthetic component, comprising:
selecting a first rotation block from a plurality of rotation blocks, the plurality of rotation blocks each having first and second upper surfaces and a lower surface disposed opposite the first and second upper surfaces, the first and second upper surfaces being offset relative to each other a distance extending in a direction transverse to the lower surface, the distance differing between each of the plurality of rotation blocks, the first and second upper surfaces defining an offset axis having a fixed angular orientation relative to an axis defined by the lower surface, the fixed angular orientation differing between each of the plurality of rotation blocks;
inserting the first rotation block into a gap between a resected proximal surface of a tibia and unresected condylar surfaces of a femur such that the lower surface contacts the resected proximal surface of the tibia, the first upper surface contacts a first unresected condylar surface of the femur, and the second upper surface contacts a second unresected condylar surface of the femur; and
evaluating correspondence between the offset axis of the first rotation block with a condylar axis defined by the first and second condylar surfaces.

2. The method of claim 1, further comprising evaluating collateral ligament tension while the first rotation block is disposed within the gap.

3. The method of claim 2, further comprising selecting a first spacer from a plurality of spacers if it is determined that the collateral ligament tension is too loose, the plurality of spacers each having an upper surface and a lower surface and a thickness defined therebetween, the thickness differing between each of the plurality of spacers by predetermined increments.

4. The method of claim 3, further comprising:
connecting the first spacer to the first rotation block such that the upper surface of the first spacer is positioned adjacent the lower surface of the first rotation block and so that the lower surface of the first spacer assumes the lower surface of the first rotation block for contact with the resected proximal surface of the tibia,
inserting the first rotation block and spacer into the gap, and
reevaluating the collateral ligament tension while the first rotation block and spacer are disposed within the gap.

5. The method of claim 4, wherein the connecting step includes one of inserting pegs of the first spacer into corresponding openings in the rotation block and sliding a dovetail of the first spacer into a correspondingly shaped groove extending into the lower surface of the first rotation block.

6. The method of claim 4, further comprising disconnecting a second spacer of the plurality of spacers from the rotation block prior to connecting the first spacer to first the rotation block.

7. The method of claim 1, selecting a second rotation block from the plurality of rotation blocks and repeating the inserting and evaluating steps when it is determined that there is no correspondence between the offset axis of the first rotation block and the condylar axis.

8. The method of claim 1, further comprising calibrating a bone preparation instrument based on the fixed angular orientation when it is determined that there is correspondence between the offset axis and condylar axis.

9. The method of claim 1, further comprising:
connecting a handle to the first rotation block prior to inserting the first rotation block into the gap, and
disconnecting the handle from the first rotation block once the first rotation block is disposed in the gap.

10. The method of claim 1, wherein the condylar axis is a posterior condylar axis of unresected posterior condyles.

11. The method of claim 1, wherein the condylar axis is a distal condylar axis of unresected distal condyles.

12. The method of claim 1, wherein the lower surface and the first and second upper surfaces of each of the plurality of rotation blocks are planar.

13. The method of claim 12, wherein the first and second upper surfaces of each of the plurality of rotation blocks are planar.

14. The method of claim 12, wherein the first and second upper surfaces of each of the plurality of rotation blocks have a surface area defined by a standard deviation of a dataset comprised of distances between tangent points of lateral and medial condyles of a population of femurs.

15. The method of claim 14, wherein the inserting step includes inserting an intermediate portion into an intercondylar notch of the femur, the intermediate portion being positioned between the first and second upper surfaces and sitting proud of the first and second upper surfaces.

16. A method of preparing a femur for receipt of a prosthesis, comprising:
selecting a first rotation block from a plurality of rotation blocks, the plurality of rotation blocks each having first and second upper surfaces and a lower surface disposed opposite the first and second upper surfaces, the first and second upper surfaces being offset relative to each other a distance extending in a direction transverse to the lower surface, the distance differing between each of the plurality of rotation blocks, the first and second upper surfaces defining an offset axis having a fixed angular orientation relative to an axis defined by the lower surface, the fixed angular orientation differing between each of the plurality of rotation blocks;
selecting a first spacer from a plurality of spacers, the plurality of spacers each having an upper surface and a lower surface and a thickness defined therebetween, the thickness differing between each of the plurality of spacers by predetermined increments;
connecting the first spacer to the first rotation block such that the upper surface of the first spacer is positioned adjacent the lower surface of the first rotation block;
inserting the first rotation block into a gap between a resected proximal surface of a tibia and unresected condylar surfaces of a femur such that the lower surface of the first spacer contacts the resected proximal surface of the tibia, the first upper surface of the first rotation block contacts a first unresected condylar surface of the femur, and the second upper surface of the first rotation block contacts a second unresected condylar surface of the femur;
evaluating gap tension and correspondence between the offset axis of the first rotation block with a condylar axis defined by the first and second condylar surfaces,
if the gap tension was determined to be too loose or too tight, selecting a second spacer from the plurality of spacers and repeating the connecting, inserting, and evaluating steps, and
if the condylar axis was determined to not correspond with the offset axis of the first rotation block, selecting a second rotation block and repeating the connecting, inserting, and evaluating steps; and
resecting the first and second condylar surfaces along a resection plane at an angle from the condylar axis substantially equal to the fixed angular orientation of one of the rotation blocks determined to have a corresponding offset axis with the condylar axis.

17. The method of claim 16, wherein the resecting step includes resecting the first and second condylar surfaces at a depth corresponding to a combined thickness of a finally selected spacer and rotation block.

18. A method of preparing a femur for receipt of a prosthesis, comprising:
selecting a static assessment device from a plurality of static assessment devices, each of the static assessment devices having first and second femoral contact surfaces positioned at different elevations from a bottom surface of the static assessment device;
inserting the static assessment device into a gap between a femur and tibia such that the bottom surface of the static assessment device contacts a resected surface of a proximal tibia, the first femoral contact surface contacts a first condylar surface, and the second femoral contact surface contacts a second condylar surface, the first and second femoral contact surfaces having a fixed relationship relative to the bottom surface while disposed within the gap;
determining a condylar angle of a condylar axis defined by the first and second condylar surfaces based on a known offset distance between the first and second femoral contact surfaces; and
resecting the first and second condylar surfaces along a resection plane based on the determined condylar angle.

19. The method of claim 18, wherein the static assessment device comprises a rotation block and a spacer removeably connected to the rotation block, the rotation block comprising the first and second femoral contact surfaces, and the spacer comprising the bottom surface.

20. The method of claim 18, further comprising:
connecting a handle to the static assessment device prior to inserting the first rotation block into the gap, and
disconnecting the handle from the static assessment device once the first rotation block is disposed in the gap.
